(19) United States
(12) Patent Application Publication
(10) Pub. No.: US 2004/0176749 A1
(43) Pub. Date: Sep. 9, 2004

(54) Insert for the treatment of dry eye

(76) Inventors: Chris P Lohmann, Regensburg (DE); Achim Goepferich, Sinzig (DE)

Correspondence Address: Paul J Vincent, Dreiss Fuhlendorf Steimle & Becker, Postfach 10 37 62, Stuttgart, D-70032, DE

(21) Appl. No.: 10/483,047

(22) PCT Filed: Jul. 12, 2002
(86) PCT No.: PCT/EP02/07805

(30) Foreign Application Priority Data
Jul. 12, 2001 (DE) 101338708

(51) Int. Cl. A61F 2/02

(52) U.S. Cl. 604/891.1; 424/425

(57) ABSTRACT

The invention relates to an insert for the treatment of dry eyes, wherein the insert can be placed on or inserted into the lacrimal sac or the cornea.

The invention concerns an insert for the treatment of dry eye, the production thereof as well as the use of an insert for dispensing proteins or peptides into the eye, in particular growth factors and, for example, epidermal growth factor (EGF) for the treatment of so-called “Dry Eye Syndrome”.

The following description explains the invention by way of example using EGF as a possible active ingredient selected from the group of proteins and peptides, in particular growth factors, which can be utilized within the context of the invention. The invention is, however, not intended to be limited to EGF. All other kinds of proteins and/or peptides can be utilized, in particular, growth factors.

In comparison to a healthy person, patients who are suffering from dry eye syndrome have reduced production of epidermal growth factor (EGF) in the epithel cells of the cornea compared to the normal physiological level.

Approximately 50 years ago, Henrik Sjögren described a disease caused by autoimmune damage to the issue of the tear gland resulting in reduced tear secretion and disease of the surface of the eye. He named this disease keratoconjunctivities sicca (KCS). In the meantime, it has been discovered that KCS or “Dry Eye” is one of a plurality of diseases which are associated with reduced tear secretions or increased evaporation of the tear film.

In Cornea 12, 202, 1994, Tsubota et. al. have classified patients having dry eye disease into three groups: (1) the dry eye in conjunction with Sjögren syndrome, (2) autoimmune positive dry eyes (circulating antibodies) and (3) simple dry eye (any kind of malfunction of the eye surface, not necessarily in combination with an autoimmune disease).

Dry eye disease typically causes eye irritation similar to that caused by foreign bodies in the eye as well as eye pain, eye burning and the like.

The clinical symptoms of KCS result from the pathological changes in the epithel surface of the cornea in KCS patients. It is well known that the eptihel cornea of KCS patients have a plurality of differences compared to the epithelium of healthy patients including: (1) the normal epithet metabolism is altered (2) there is unevenness in the thickness of epithel layer (3) the surface of the epithel is irregular (4) there is a lack of sufficient intercellular binding such as hemidesmosomes, which are not present in sufficient quality and quantity.

In modern times, dry eye disease is usually treated with so-called oily eye drops or inserts which dissolve slowly, also referred to as artificial tears. In most cases, the success of the treatment is inadequate. Among other reasons, this lack of success is due to the short dwell-time of these types of pharmaceutical materials in the eye. Eye pharmaceuticals for the treatment of dry eye syndrome were developed for the first time during the 1970s in the form of polymers in watery solutions such a polyvinylalcohol, cellulose derivatives or polyvinylpyrrolidone or other polymers due to their oily effects (U.S. Pat. No. 4,120,949, U.S. Pat. No. 4,744,980; U.S. Pat. No. 4,883,658; U.S. Pat. No. 5,209,927, and U.S. Pat. No. 5,770,628).

Moreover, “artificial tears” have also been developed having matrices consisting essentially of water soluble polymers which slowly dissolve after placement into the lacrimal sac (U.S. Pat. No. 4,343,738).

In addition to solutions and solid inserts, semi-solid preparations have also been developed, including polymers, which produce a certain oily effect (U.S. Pat. No. 5,075,104).

Another point of departure for treating dry eye syndrome was the development of substances stimulating the production of tears (U.S. Pat. No. 4,820,737). In addition, other pharmaceutical materials such as retionoids (U.S. Pat. No. 4,826,871; U.S. Pat. No. 4,966,773; and U.S. Pat. No. 5,185,372) as well as calcium salts (U.S. Pat. No. 5,290,572; U.S. Pat. No. 5,595,764) and steroids (U.S. Pat. No. 5,041,434) have been tested for the treatment of the disease. None of these attempts lead to a significant improvement in the changes in the cornea which have been described above. They only permit brief, temporary treatment of the symptoms which result from damage.

Similar problems related to the short time duration during which the eye therapy is effective result when a pharmaceutical is to be dispensed to the eye. Eye drops only lead to a very short time period of approximately 30 seconds during which they are present in the eye. In order to overcome this problem, a pharmaceutical was developed in the early 1970s which dispensed pharmaceutical material over a long period of time. The inserts were disposed in the lacrimal sac and were usually made from materials which did not degrade and/or decompose and/or dissolve during the treatment. It was therefore necessary to remove them from the eye after a certain period of time (U.S. Pat. No. 3,618,604; U.S. Pat. No. 4,057,619). Some of the systems were designed in such a fashion as to simplify remove of the materials, by way of example, by providing materials which could be withdrawn using magnets (U.S. Pat. No. 3,626,940). Due to the use of solid carrier materials, the release mechanism for the pharmaceutical was limited to diffusion. The rate of release could therefore be controlled through use of micro porous materials (U.S. Pat. No. 3,828,777). In addition, biologically degradable materials were utilized for inserts which dissolved during use in the eye (U.S. Pat. No. 3,867,519; U.S. Pat. No. 4,179,497). These inserts had the advantage that removal from the eye was no longer necessary after completion of the treatment.

Further developments included the use of lipids instead of polymers as the material for use with inserts (U.S. Pat. No. 3,968,201) as well as the improvement and adjustment of the geometry and the mechanical properties to improve the release (U.S. Pat. No. 3,963,025) as well as special inserts with reinforced units (U.S. Pat. No. 5,395,618).

In contrast thereto, it is the object of the present invention to improve treatment of dry eye patients.

This object is achieved through the features of patent claims 1, 16 and 17.

It has thereby been determined that EGF represents an effective therapeutic material for eye treatment. In accordance with the invention, pharmaceuticals have been developed which increase the EGF levels in the eye and which continue to release significant levels of EGF and calcium with a preferably linear kinetic, over a time period of many hours. This is necessary in order to stimulate the epithel cells of the cornea to synthesize hemidesmosomes and other intercellular adhesion molecules and to thereby facilitate cell-cell-binding.

Unexpectedly, it is turned out that these pharmaceuticals are capable of significantly improving the therapy of patients having dry eyes. The invention also concerns a dispensing system for medication which permits improved introduction of EGF into the eye.

Cytokines control the architecture, the normal physiological activity and, if necessary, the wound-healing processes of cornea epithets. Among a plurality of cytokines, EGF appears to be extremely important with regard to the physiological functions of the cornea epithelium mentioned above. EGF is responsible for the maintenance of the cornea epitheliums and is produced in the tear glands and in the basal cells of the cornea epithets. The EGF receptors of the eye are located in the cornea epithel, the lens, as well as in the cornea endothel. The receptors have a high affinity to EGF and can be saturated. When the eye is injured, the density of the epithel EGF receptors increases. For this reason, EGF is proposed for the treatment of the injured eye. Individual serum preparations which include a plurality of other cytokines other than EGF have, in the meantime, been tested with regard to dry eye syndrome therapy.

In dry eye patients, the level of EGF in tears and in the cornea epithet cells is significantly lower than in normal patients. Towards this end, samples of tear liquid have been taken. The samples have been extracted using the capillary effect. The capillary tubes were introduced at an angle of 10 to 30 degrees above the horizontal axis and at an angle of 10 to 40 degrees with respect to the surface of the lower fornix. The tip of the tube is brought into contact with the surface of the tear liquid and is slowly guided on the fornix inferior from the middle line towards the side corner of the eye. One must thereby be careful not to come in contact with the surface of the eyeball in the event of blinking, and the tube is quickly removed from the fornix. All samples were directly transferred into Eppendorf tubes, frozen with dry ice, and stored at −70 degrees prior to determination of the EGF concentration. In order to compare various individual samples, the time of the sample extraction was recorded and correlated with the EGF content. The EGF concentration in the tear fluid is determined by utilizing ultra-sensitive immunofluorometrical ELISA tests of the sandwich type (R&D Systems, Minneapolis, USA). In this two-stage, solid phase technique, immunoreactive EGF is initially bound to a polyclonal anti-EGF antibody which attaches to the solid phase and which can be quantified using a monoclonal anti-human EGF antibody. The smallest amount of EGF which can be determined using this technique is 0.2 pg/ml for non-thinned samples and 40 pg/ml for thinned samples.

The following table shows the results of analyzed EGF amounts in the eye fluid of normal patients and dry eye patients.

TABLE 1

| | |
|---|---|
| Healthy (n = 30) | 1.7-4.3 ng/ml |
| Patients with primary KCS (n = 30) | 0.02-0.03 ng/ml |
| Patients with Sjörgren's syndrome (n = 30) | 0.02-0.22 ng/ml |
| Patients with meibomitis (n = 30) | 0.3-0.95 ng/ml |

In further experiments, cornea epithel cells for molecular biological investigations have been examined with regard to the individual manufacture or synthesis of EGF by measuring the EGF mRNA. A small piece of tissue having a diameter of 0.8 mm was extracted at a separation of approximately 2 mm from the limbus. The sample was directly transferred after removal into an Eppendorf tube together with 300 μl of RNAzol (WAK Chemie, Heidelberg, Germany). The quantitative analysis of the EGF was carried out using a conventional technique with which mRNA for epidermal growth factors has been intensified with PCR (Polymerase Chain Reaction) and then analyzed. Table 2 shows the results of the EGF mRNA levels which were analyzed in the cornea epithet cells of normal people and of patients with dry eye.

TABLE 2

| | |
|---|---|
| Healthy (n = 30) | 6.25-13.11 units |
| Patients with primary KCS (n = 30) | 0.23-1.34 units |
| Patients with Sjörgren's syndrome (n = 30) | 0.31-1.11 units |
| Patients with meibomitis (n = 30) | 2.78-6.69 units |

The above result shows that patients which are suffering from primary dry eye syndrome (primary KCS) and patients with Sjörgren's syndrome have significantly reduced EGF levels. One suspects that these lowered local EGF levels lead to changes in the cornea and eventually to dry eye syndrome.

On the basis of these results, a histological study and two clinical studies have been carried out. In the histological study, human cornea was continuously subjected to a solution containing 5% EGF for various lengths of time, the time periods ranging from 30 seconds to 4 hours. Following a contact time of 30 seconds, which approximately corresponds to the time during which eye drops remain in contact with the cornea surface, a weak epithel cell architecture has been observed, having only few intercellular adhesion molecules. In contrast thereto, following an EGF period of 4 contact hours with the cornea epithel surface, the epithel exhibits an optimal architecture with a plurality of intercellular adhesion molecules such as hemidesmosomen. In a clinical study with 5% EGF in eye drops, no positive effect on the symptoms of the patient or on the morphological indications were found in 13 patients. In contrast thereto, with continuous application of 5% EGF, dissolved in methyl cellulose, over a period of 4 hours, a significant improvement in the patient symptoms as well as in the morphological indicators of the cornea epithets have been observed over a period of 5 to 7 days.

EGF induces the synthesis of cell-to-cell adhesion molecules and is therefore a reasonable active ingredient for the utilization and treatment of dry eye patients. A substantive aspect of the invention is therefore to develop a pharmaceutical release system which permits EGF-deficient patients to receive this type of growth factor or other proteins and/or peptides, in particular growth factors.

Although the local release of EGF in the eye is the primary point of departure, in the future it may turn out to be reasonable to systematically dispense EGF over a plurality of differing introductory paths (oral or parenteral). Is it moreover conceivable to modify the cells of the tear gland or stem cells of the cornea epithets in such a fashion that they produce EGF or to transplant EGF-producing cells.

Within the context of this invention, the expression “EGF” preferentially refers to human EGF. However, EGF from other living entities or substances which bind to EGF receptors or chemical modifications of these types of substances, for example PEG related substances may also be used. Human EGF is a 6.045 kD polypeptide chain having 53 amino acids with three disulfide compounds within the chain. In the past, EGF has only been proposed as a therapeutic agent for promoting the healing of wounds, in particular, following cornea and refractive surgery.

It is therefore the object of the present invention to introduce a carrier system for the dispensing of EGF at its location of use. Of particular importance is the continuous dispensing of EGF to the cornea epithel surface.

Ophthalmic agents which can be utilized in order to locally release EGF to the eye include drops, creams, gels or solid inserts. These types of preparatives contain the active component EGF in the range 0.0000001 to approximately 20% by weight, preferentially between 0.0001 to approximately 1% by weight and particularly preferred 0.01 to 0.5% per weight or in such an amount that the EGF level in the tear liquid is raised to the physiological level. The dosing of the active components can depend on various factors such as the manner in which the dispensing is carried out, need, age or personal conditions.

The dispensing of eye drops can, in principle, be facilitated in two different ways: The dissolving of EGF in water or the application as a suspension, e.g. in oily carriers.

There are a plurality of additives which can be utilized to produce drops. Examples of conventional pharmaceutically acceptable carriers and additives are discussed below. They include a carrier, isotonic agents, buffer solutions, complex builders, solvents and thickening agents. Examples of these types of carriers and additives can also be extracted from WO 91/15206 or from the pharmaceutical literature.

For the production of watery EGF solutions, the active components are dissolved in a sterile watery isotonic solution and, to the extent necessary, buffered to the desired pH value.

An opthalmicum which includes EGF normally comprises a carrier.

Possible carriers are, in particular, gels, gum tragaconth, methyl cellulose and/or polyvinylpyrrolidons, agar or alginic acid or their salts such as sodium alginate, acacia gum, polyvinylpyrrolidon and polyethylyne glycol. For dispensing using drops, e.g. watery solutions of EGF or, for example, suspensions of EGF in oils, are suitable. Suitable lipophile solvents or carriers include fatty oils, for example sesame oil or synthetic fatty esters, for example, ethyloleat or triglycerides or watery injection suspensions containing viscosity raising substances, for example sodium carboxymethyl cellulose, sorbitol and/or dextrin and also include stabilizers to the extent desired.

Additional carriers include water or mixtures of water and water mixable solvents for example C₁- to C₇-alcohols, plant oils or mineral oils having between 0.5 and 5% by weight of hydroxyethyl cellulose, ethyloleat, carboxymethyl cellulose, polyvinylpyrrolidon or non-toxic water soluble polymers for ophthalmic applications such as e.g. cellulose derivatives, such as methyl cellulose, alkali metal salts of carboxymethyl cellulose, hydroxymethyl cellulose, hydroxyethyl cellulose, methylhydroxypropyl cellulose, hydroxypropy cellulose, chitosan, scleroglucan, acrylate or metharylate for example the salts of polyacrylic acids or ethacrylate, polyacrylamide, natural products such as gels, alginate, pectin, traganth, karaya gum, xanthan gum, carrageen, agar and acacia gum, starch derivatives such as starch acetates, hydroxylpropyl starches or also other synthetic products such as poloxamer, e.g. poloxamer F127, polyvinyl alcohol, ployvinylpyrrolidon, polyvinylmethylether, polyethylenoxide, preferentially cross-linked polyacryl acids, for example neutral carbopol or mixtures of these polymers. The preferred carrier is water. Cellulose derivatives can, for example, be methyl cellulose, alkali metal salts of carboxymethyl cellulose, hydroxymethyl cellulose, hydroxyethyl cellulose, methylhydroxypropyline cellulose and hydroxypropyline cellulose, neutral carbopol or mixtures thereof. The concentration of the carrier can assume values between 0.1 and 100,000 times the concentration of the active components.

Solvent agents and other auxiliary materials can also be utilized in an ophthalmicum and include, for example, tyloxapol, fatty acid glycerol-polyethylene-glycol ester, fatty acid polyethylene-glycol ester, polyethylene glycol, polyglycerolester, polysorbate 20, polysorbate 80 or mixtures of these components. A particular example for a solvent is the reaction product from rhicinus oil and ethyl oxide, e.g. the commercially available product Cremophor EL® or Cremphor RH 40®. Reaction products of rhicinus oil and ethyl oxide have turned out to be particularly good solvents which are particularly well accepted by the eye. Another preferred solvent is tyloxapol. The concentrations utilized depend, in particular, on the concentration of the active components. The amount which is introduced is typically sufficient to dissolve the active components. For example, the concentration of the solvent can be between 0.1 to 5,000 times the concentration of the active components.

Electrolytes which are interesting for the composition include substances which permit adjustment of the pH value during storage or use of the formula as well as electrolytes which influence the protein structure and therefore the stability of EGF. Examples of buffer solutions include acetate, ascorbate, borate, hydrogen carbonate/carbonate, citrate, gluconate, lactate, phosphate, propionate and TRIS buffer. Tromethamine and borate buffers are the preferred buffer solutions. The introduced amounts of buffer solutions can, for example, correspond to the amount which is necessary in order establish and maintain a physiologically tolerable pH level. The pH level is typically in the range between 5 to 9 and preferably between 6 to 8.5 and particularly preferably between 6.5 and 8.2.

Moreover, electrolytes can influence the osmotic pressure and the protein conformation. Such electrolytes include, for example, ionic components, for example alkali salts or alkaline earth halogens, such as CaCl₂, KBr, KCl, LiCl, NaI, NaBr or NaCl, Na₂SO₄, or boric acid. Non-ionic agents which increase the tonicity are for example urea, glycerol, sorbitol, mannitol, propylenglycol or dextrose. The tonicity enhancing agents may be introduced in an amount which leads to a useable ophthalmic preparation having an osmolality of between approximately 50 to 1000 mOsmol and preferentially between 100 to 400 mOsmol, particularly preferably between 200 to 400 mOsmol and advantageously between 250 to 350 mOsmol.

Examples for preservatives include quaternary ammonium salts such as sepazonium chloride, cetyhltrimethylammonium bromide (cetrymide), cetylpyridinium chloride, benzoxomium chloride, benzethonium chloride, domiphenbromide (Bradosol®) or benzalkonium-chloride, alkyl mercury salts of the thiosalicyl acids, such as thiomersal, phenyl mercury nitrates, phenyl mercury acetates or phenyl mercury borates, parabene, for example, methyl parabene or propyl parabene, alcohol such as chlorobutanol, benzylalcohol or phenylethalnol, guanidine derivatives such as chlorohexadine or polyhexamethlyen-biguanid, sodiumpercarbonate, Germal® or sorbine acids. Preferred preservative agents include quaternary ammonium salts, alkyl mercury salts and parabene. Where indicated, a sufficient amount of preservatives can be added to ophthalmika in order to assure protection with respect to secondary contaminations during use caused by bacteria and fungus.

Moreover, ophthalmika can contain additional non-toxic carriers, for example moisturizing agents or filling agents which, for example, contain polyethylenglycol or derivatives thereof (for example methylPEG or PEGamine) having a molecular weight between 200 to 10,000 or more. Additional carrier agents which can be utilized, if desired, are discussed below, wherein these examples are not intended to limit the possible numbers of carrier materials. Preferentially such materials include complex builders for example disodium-EDTA or EDTA, antioxidants such as ascorbic acid, acetylcystein, cystein, sodium hydrogen sulfide, butyl-hydroxyanisol, butylhydroxytoluen or alpha-tocopherol-acetate, stabilizers such as thiourea, thiosorbitol, sodium-dioctyl-sulfocussinate or monothioglycerol or other carriers such as, for example, laurin acid sorbitolester, triethalnolaminoleat or palm acid esters. Preferred carriers are complex builders such as disodium-EDTA. The amount and type of added carriers depends upon the particular requirements and is generally in the range between approximately 0.0001 to approximately 90% by weight.

EGF can be dispensed into the eye using a small device which can be inserted into the lacrimal sac. These types of systems release proteins or peptides, in particular growth factors such as EGF, over a period of time between one hour and two weeks. A particularly preferred period of release time is between four hours and one week. The shapes and sizes of these types of inserts are accommodated to the anatomy and physiology of the eye. The inserts can, e.g. have a geometry which is cylindrically round or oval or any other shape which is suitable to be inserted into the lacrimal sac of the eye. Moreover, the insert can also have the shape of a contact lens which, in this case, can be placed onto the cornea. The insert is preferably round or oval and has a first diameter of r₁ between 0.1 mm and 20 mm and a second diameter r₂ between 0.1 mm and 20 mm as well as a thickness d between 1 μm and 5 mm. Oval geometries are particularly preferred having a radius r₁=0.5 mm to 18 mm, r₂=0.5 mm to 10 mm and a thickness d=10 μm to 1 mm. An additional important characteristic is that all inserts dispense calcium ions together with EGF, since it has been discovered that calcium is necessary for improvement of the cell-to-cell contact of the corneal epithel.

EGF can be dispensed from thin films which, for example comprise polymer films in which EGF is embedded. EGF can be dissolved or suspended in the matrix. Films of this type have the advantage of being easy to produce and large amounts of them can be cut out as individually inserts or punched out in the desired geometry from a larger sheet.

One layer films made from EGF have only one single matrix material or matrix material mixture in which the EGF is embedded. A simple manner for producing these types of matrices for local EGF release is to disburse proteins in a solution or in a melted matrix material. Evaporation of the solvent or cooling of the melt leads to an EGF-enriched matrix. An example of this embodiment is hydrogels. EGF can, for example, be dissolved in a 1% (w/w) watery alginate solution. Following shaping in molds with a defined surface geometry, the hydrogel is dried in order to form a solid film from which the insert can be fashioned through cutting or punching of pieces with the desired geometry. The thickness of the film can be varied through increase of the alginate content or through adjustment of the mold area.

In dependence on the matrix material chosen, the inserts can be decomposable or non-decomposable. For decomposable inserts, there is no need for the insert to be removed following use. Materials which can be used for the production of non-decomposable inserts include e.g. polyacrylates or ethylenvinylacetate copolymers. Such matrices can be charged with water soluble substances in addition to EGF which act as porogenes in order to facilitate control of the release of EGF. In the case of non-decomposable materials, the release of EGF can be controlled through diffusion and can be influenced by the type and the amount of porogenes. Porogene materials are, e.g. water soluble polymers such as for example polyethylenglycol, various polysaccharides or proteins such a collagen or gelatin. Other water soluble substances of low molecular weight can fulfill the same purpose.

Materials which can be utilized for the production of a decomposable system are e.g. water soluble polymers having matrices in solid or semi-solid form which dissolve in the liquid environment of the eye or which soften or melt at body temperature. Examples of such polymers include alginate or cellulose derivatives such as for example methyl cellulose. The preferred material from which decomposable inserts can be fashioned is alginate which consists essentially of α-L-guluron acid and β-D-mannuron acid. The fraction of monomers can be selected in order to control properties such as mechanical stability and pharmaceutical release rate. Due to the presence of carboxyl acid groups, the alginates are charged and can be cross-linked through the addition of cations such as calcium or magnesium. The degree of cross-linking permits the release rate of EGF as well as the erosion stability of the insert to be controlled. A particular case of decomposable polymers are polymers which are subjected to hydrolysis in a watery environment and which decompose into water-soluble monomers and oligomers. Substances which decompose, soften, or melt under thermal changes are e.g. fats such as triglycerine or phospholipide.

A plurality of films can be combined in order to reduce the protein release rate from the films or to control the protein release rate to achieve e.g. a more linear release kinetics. Such laminates have at least two layers which differ from each other either with respect to the matrix material from which they are made and/or their individual EGF charge.

An example for a laminate of this type is a structure which consists essentially of a central layer of high EGF content which is covered on both sides by film layers having a lower EGF content. Other types of laminates could comprise a sandwich-like structure having alternative sequences of EGF-charged and EGF-free layers.

The laminates can e.g. be produced through impregnation with or spraying on of various matrix solutions, one upon the other, through coacervation or through dipping of pre-fabricated films in appropriate solutions or material melts which are to be introduced to the laminate. In the latter case, the polymer solutions are produced from polyelectrolytes having opposite charges. The charge interactions lead to the deposition of polymer chains. Bound EGF is therefore immobilized in the resulting polymer matrix. Moreover, the growth factor, such as EGF, is also stabilized by the matrix and dispensed in stable form during use. No activity loss therefore occurs during production, storage and/or application of the active material. In order to achieve decomposable systems, at least one of the polyelectrolytes must be a degradable polymer. Alternatively, a third component which is water soluble or biologically decomposable is added to the mixture. Examples of polyelectrolyte combinations are sodium alginate/chitosan, sodium aginate/gelatin and acacia gum/gelatin.

Instead of films and laminates, reservoir systems can be used for the controlled release of EGF from an insert. Reservoir systems comprise, for example, a liquid or semi-solid EGF dispersion which is surrounded by a membrane through which the EGF release rate can be controlled. Further examples of EGF reservoirs are solid, water soluble mixtures which can be produced, by way of example, by freeze-drying and which dissolve after introduction of the insert into a liquid or semi-solid system.

The membranes controlling the release rate can, for their part, be decomposable polymers such as e.g. alginate which is cross-linked with calcium ions in order to increase its stability. Non-decomposable micro porous polymers can be utilized as a diffusion barrier to control the release of EGF from the reservoir.

Moreover, films, laminates or reservoir systems can be utilized in connection with the additional carriers in order to control the release of EGF. Particles of less than 100 μm size, for example micropheres, nano particles or liposomes can be charged with EGF in order to control the release rate. When particles of this type are directly dispensed in the eye they usually disappear quickly. By embedding them in an insert such as, for example, a film, a laminate or a reservoir-like device it is possible to overcome this problem. During production, systems of this type are simply disbursed in a matrix solution or within the material melt which is utilized for the production of the insert. These particular systems can be equally successfully bound in films, laminates or reservoir systems.

Moreover, there are a number of additional release systems which can be utilized for local dispensing of EGF from an eye insert which, however, cannot be classified as a film, laminate or reservoir system.

In situ-gelling systems are liquid preparations prior to use in the eye and can therefore be easily administered by a patient. When they are introduced into the lacrimal sac, their viscosity increases or they solidify and thereby build an EGF enriched deposit. Examples of systems of this type include poloxamer solutions which gel when subjected to a temperature increase. A further example are polyelectrolytes which can change their charge in response to pH changes such as, for example, polyacrylate and therefore precipitate in a watery environment.

Moreover, EGF can be dispensed from micro chips which open a small reservoir to release the pharmaceutical material in pre-programmed time intervals and durations. For example, the release can be effected through a programmable read-only memory (EPROM) on the same micro chip (U.S. Pat. No. 5,797,898). It is moreover possible to couple such a dispensing system to a biosensor which measures the local EGF level and maintains the output of a adjustable dispensing unit.

Finally, osmotic pumps can also be utilized.

Osmotic pumps have, for example, a EGF reservoir which holds an osmotically active substance in addition to the EGF dose and which is surrounded by a semi-permeable membrane. When such a system is inserted into the eye, water begins to pass through the semi-permeable membrane and begins to dissolve or thin out the EGF and the osmotically active substance. Through the increase in osmotic pressure, the EGF is pushed out of the reservoir. EGF thereby leaves the system through the membrane which is, for example, micro porous or has small openings.

The invention is described below with embodiments and drawings.

### EXAMPLE

### Production of an EGF-Containing Alginate Film

1. Production of an Alginate Gel:

1 g of sodium alginate (protanal SF 120, Provona Biopolymer, Norway) is precisely weighed in a 100 ml tube having a cover. 99 g of a sterile intra-ocular washing solution (BSS sterile intra-ocular solution Pharmacia & Upjohn, Holland) is added thereto. During swelling of the alginate powder, the mixture is carefully stirred using a magnetic stirrer. After the alginate has dissolved, 150 mg CaCl₂×2H₂O is added and the gel is stirred for at least an additional 24 hours until it is clear or opaque and free of particles.

2. Production of an EGF Solution:

1 mg EGF (rEGF, Biomol, Hamburg, Germany) is dissolved in 1 ml of doubly distilled water. After complete dissolution, an additional 9 ml of water are added.

3. Production of an EGF Charged Alginate Film:

5.50 g alginate gel are weighed in a petri dish (7 cm diameter, 4 cm in height). 1 ml of EGF solution (EGF content: 100 μg/ml) is mixed into the gel while stirred by a glass rod. The resulting mixture is added to a Teflon mold (4 cm diameter, 5 cm in height). The film is dried for 48 hours at ambient conditions in a working station using a laminar air film.

4. Cross-Linking of the Films and Manufacturing of the Insert:

The dried EGF-charged alginate film is removed from the Teflon mold and dipped for 60 seconds into a 5% (w/w) watery calcium chloride solution. The film is dried in a working station for a period of 24 hours using a laminar air film. Oval inserts (r₁=9 mm in length and r₂=4 mm) are cut or punched out of the film having a final thickness of approximately 100 μm.

Possible embodiments of the inserts are shown below with regard to the drawing.

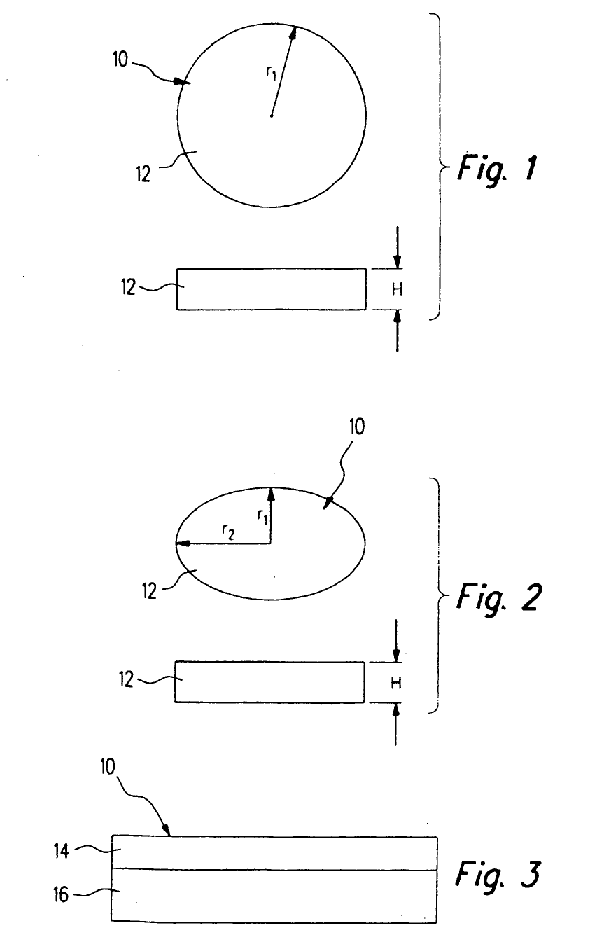

FIG. 1 shows a round insert,

FIG. 2 shows an oval insert,

FIG. 3 shows a two-layered insert,

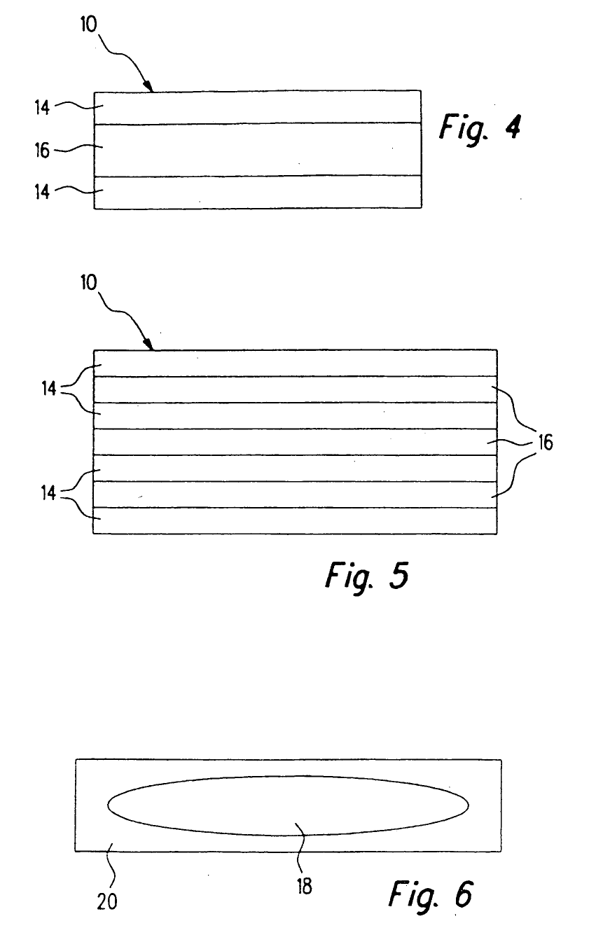

FIG. 4 shows a three-layered insert,

FIG. 5 shows an insert with EGF charged and EGF free layers,

FIG. 6 shows a reservoir-like device,

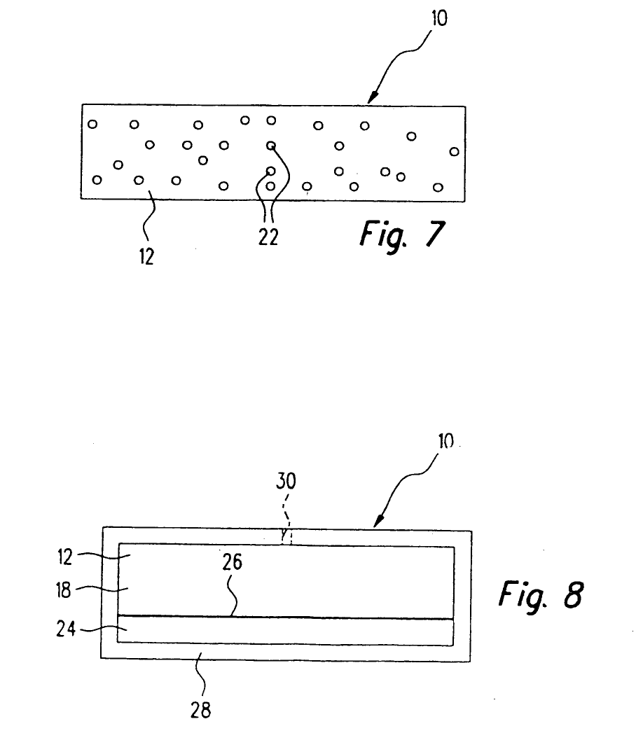

FIG. 7 shows an additional insert, and

FIG. 8 shows an insert in the form of an osmotic pump.

FIGS. 1 and 2 show two differently shaped inserts 10, namely a round as well as an oval one. The inserts are cylindrical and have a height H=1 mm. EGF is embedded in the matrix material 12. The round insert 10 thereby has a radius r₁=1 mm. The oval insert has a radius r₁=1.5 mm and a radius r₂=2 mm.

FIG. 3 shows an insert 10 which, by way of example, can have the shape of FIGS. 1 and 2 and is made from essentially two differing layers 14, 15, wherein the upper layer 14 is EGF free and the lower layer 16 has charge of EGF.

FIG. 4 describes a similar structure, wherein here the EGF-charged layer 16 is enclosed by two EGF-free layers 14. In consequence thereof, the release of EGF to the eye in the lacrimal sac can be better controlled after placement of the insert 10 therein.

FIG. 5 shows an augmented structure in which EGF-free layers 14 and EGF-charged layers 16 alternate, wherein three EGF-charged layers 16 are embedded between the EGF free layers 14.

FIG. 6 shows a reservoir system as described above, wherein the reservoir 18 having an EGF containing solution is enclosed by a membrane 20 which controls and determines the release of EGF to the eye. Another possible method of embedding the EGF in a matrix 12 is by dispensing EGF-charged particles 22 in a matrix 12, wherein the matrix 12 then controls release of the EGF (FIG. 7).

The last FIG. 8 shows an additional embodiment of an insert 10. The insert is configured as an osmotic pump 10 and has an EGF-charged matrix as well as an osmotic actively substance 24, wheren the EGF-charged matrix 12 is separated from the osmotically active substance 12 by means of an impermeable membrane 26. The EGF-charged matrix 12 and the osmotically active substance 24 are commonly surrounded by a semi-permeable membrane 28. When the insert 10 is introduced into the lacrimal sac, water penetrates through the semi-permeable membrane 28 and begins to thin the EGF and the osmotically active substance 24. The resulting increase in osmotic pressure causes the EGF to be pushed through an opening 30 in the semi-permeable membrane 28 and out of the reservoir 18 to thereby gain entrance to the lacrimal sac and thereby the eye.

1. Insert for the treatment of dry eye, wherein the insert can be inserted into the lacrimal sac or can be placed onto the cornea and in which proteins and/or peptides are embedded in a manner which allows their release into the eye, wherein the protein and/or peptide are, in particular, growth factors, preferably epidermal growth factor (EGF) and/or cytokine.

2. Insert according to claim 1, wherein the insert includes a matrix charged with proteins and/or peptides, wherein the proteins and/or peptides are preferably dissolved or suspended in the matrix.

3. Insert according to claims 1 or 2, wherein the proteins and/or peptides are present in the matrix in a stable form and can be released thereby in stable form.

4. Insert according to claims 2 or 3, wherein the matrix includes releasable calcium ions.

5. Insert according to any one of the claims 1 through 4, wherein the protein and/or peptides and/or the calcium ions can be dispensed in a linear function.

6. Insert according to any one of the claims 1 through 5, wherein the insert is made from one or a plurality of layers having different protein and/or peptide contents and/or matrix materials, wherein, in particular, protein and/or peptide-charged and protein and/or peptide-free layers are provided.

7. Insert according to any one of the claims 1 through 6, wherein the protein and/or peptide are bound to particles.

8. Insert according to any one of the claims 1 through 7, wherein the insert includes a reservoir which contains proteins and/or peptides with the reservoir being enclosed in a membrane, wherein, in particular, releasable calcium ions are present in addition to proteins and/or peptides.

9. Insert according to any one of the claims 1 through 8, wherein the release of the proteins and/or peptides is effected by means of an osmotic pump.

10. Insert according to any one of the claims 1 through 9, wherein the insert contains a micro chip which controls dispensing of the protein and/or peptide out of the reservoir.

11. Insert according to any one of the claims 1 through 10, wherein the insert includes an in-situ gel system which contains proteins and/or peptides which is liquid prior to use and which gels at the application location in response to pH values or a temperature change, wherein the in-situ gelling system in the solid state, in particular, forms a protein and/or peptide containing deposit and wherein, in particular, in addition to proteins and/or peptides, dischargeable calcium ions are also present.

12. Insert according to any one of the claims 1 through 11, wherein the insert has a round or oval cylindrical shape having a diameter r₁=0.1 to 20 mm and r₂=0.1 to 20 mm and a thickness D of between 1 μm to 5 mm.

13. Insert according to claim 12, wherein r₁=0.5 to 18 mm, r₂=0.5 to 10 mm and D=10 μm to 1 mm.

14. Insert according to any one of the claims 1 through 13, wherein the insert is degradable.

15. Manufacture of an insert according to anyone of the claims 1 through 14, wherein a water soluble gel is produced, in particular an alginate gel, which is mixed with a protein and/or peptide solution, in particular a growth factor solution and, in particular, an EGF solution and a protein and/or peptide charged film is formed which is dried with inserts subsequently being cut or punched out of the film.

16. Use of an insert according to any one of the claims 1 through 14 to dispense proteins and/or peptides, wherein these growth factors are, in particular, EGF and/or cytokine, wherein the dry eye, in particular, is caused by keratoconjunctivitis, Sjögren syndrome or meibomitis.
